(19) United States
(12) Patent Application Publication
(10) Pub. No.: US 2009/0093783 A1
(43) Pub. Date: Apr. 9, 2009

(54) Use of vacuum suction in conjunction with diapers.

(76) Inventor: Mady Attila, Kihei, HI (US)

Correspondence Address: Attila Mady, 105 Manino Circle, Suite 203, Kihei, HI, 96753, US

(21) Appl. No.: 11/868,187

(22) Filed: Oct. 5, 2007

(51) Int. Cl. A61F 13/15

(52) U.S. Cl. 604/359; 604/385.01; 604/385.15; 604/385.19

(57) ABSTRACT

The application of vacuum suction to a standard diaper in order to evacuate sufficient urine and provide sufficient air movement to dry excrement sufficiently to prevent skin breakdown and permit extension of the diaper's service cycle.

The use of vacuum suction in conjunction with diapers.

### TECHNICAL FIELD

Diaper, health care, personal hygiene.

### BACKGROUND OF THE INVENTION

The population is aging. The single most labor intensive component of elder care is personal hygiene.

Personal hygiene is not only cosmetic. Urine and feces opposed to skin cause breakdown and infection. Caregiver staff are usually poorly trained, complacent and lack diligence. Reducing the amount of work required to maintain cleanliness would not only reduce costs but improve care by extending the service cycle of individual personal hygiene garments, thus requiring less input from the caregiver.

Diaper technology to date has focused exclusively on passive means such as improving the absorptive characteristics of absorptive materials. A vacuum suction approach would be effective due to the fact that the elderly do not move a great deal while in bed and also because complete evacuation of diaper contents is not necessary for clinical effect.

Complex, cumbersome and expensive machines have been proposed for the purpose of assisting with cleaning and hygiene. They all propose some manner of irrigation, which misses the point that the skin does not have to be clean, but rather dry. It also misses the fact that complex machinery is not likely to be utilized appropriately by the low-paid and poorly trained caregivers employed in many healthcare settings. Finally, aside from the issue of the exorbitant cost of the equipment itself, as well as the overhead for maintenance, this complex equipment will increase, not decrease the manpower demand on personnel.

### SUMMARY OF THE INVENTION

The application of vacuum suction to a standard diaper in order to evacuate sufficient urine and provide sufficient air movement to dry excrement sufficiently to prevent skin breakdown and permit extension of the diaper's service cycle.

### DETAILED DESCRIPTION OF THE INVENTION

A standard diaper is modified to include one or more attachments to transmit suction vacuum into the diaper. One anterior and one posterior attachment is envisioned.

The preferred embodiment is wherein standard hospital or nursing home suction is connected to this diaper. Alternatively, a modular assembly can be constructed that has a detachable reservoir for cleaning, as well as a detachable reusable power source and vacuum generator.

Vacuum levels are maintained to ensure sufficient flow to dry the skin surface. Incidental fluids drained are trapped by the reservoir. Diapers are changed on a preset schedule or as indicated by a humidity monitoring sensor.

In its preferred embodiment the diaper would be augmented with a very coarse inner layer able to disperse and retain a substantial quantity of particulate waste and still maintain high air flow rates.

There are a couple of concerns. The first issue in everyday use is that of movement. The elderly, particularly debilitated, do not move sufficiently in most cases to dislodge IV lines, transdermal patches, restraints or any other appliances' attached to their bodies. For this reason it is anticipated that the vacuum line will remain attached and will not lead to any impact on limbs or surrounding tissue.

The issue of movement might be significant in case of infants and children. For this reason it is anticipated that maximum utility of this technology would be in the elderly and otherwise disabled.

The second point is the fact that this technology does not propose to evacuate diaper contents completely. However, completely evacuation is not necessary. The intent of providing suction is to induce circulation of air over the skin and to thus reduce the humidity of the skin surface. Even adherent feces can be rapidly dried in this manner, resulting in less skin irritation and prevention of infection.

The technology is cheap, simple to use and effective. By reducing the need for caregiver vigilance, motivation and skill, this very simple technology increases the margin of safety for safe care of the patient's skin.

### DISTINCTION FROM PRIOR ART

Prior art is primarily provided for background purposes. There are no prior patents closely approximated to the current proposal.

There are a large number of patents provided to illustrate various embodiments regarding bedside commodes, mobile toilet apparatus and various bedpan enhancements. None address improvement of diapers.

USPTO U.S. Pat. No. 6,110,159, USPTO U.S. Pat. No. 5,342,583 and JP10272150 describe alternate versions of apparatus intended to irrigate the perineum in order to obtain adequate cleansing. In contrast, the currently proposed technology intends to effect desiccation through the use of suction. Purpose of suction is desiccation. No complex (and likely useless) appliances are proposed.

No closely applicable foreign art has been found. There are four Japanese patents that describe complex irrigation/drying equipment analogous to the above cited USPTO patents. Due to their complexity and lack of applicability to the current proposal, they were not cited or translated.

### DESCRIPTION OF THE DRAWINGS

FIG. 1 (longitudinal cross section) illustrates one envisioned embodiment. Individual items of interest are labeled appropriately on the diagram.

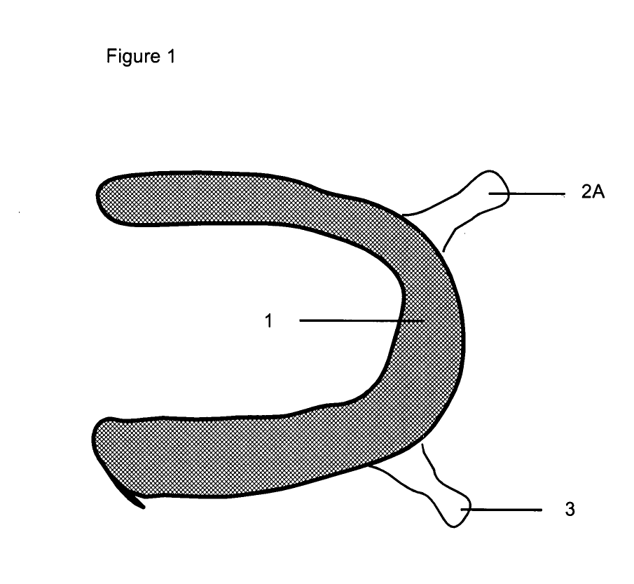

FIG. 2 (longitudinal cross section) illustrates the same embodiment and an appropriate vacuum circuit. Individual items of interest are labeled appropriately on the diagram.

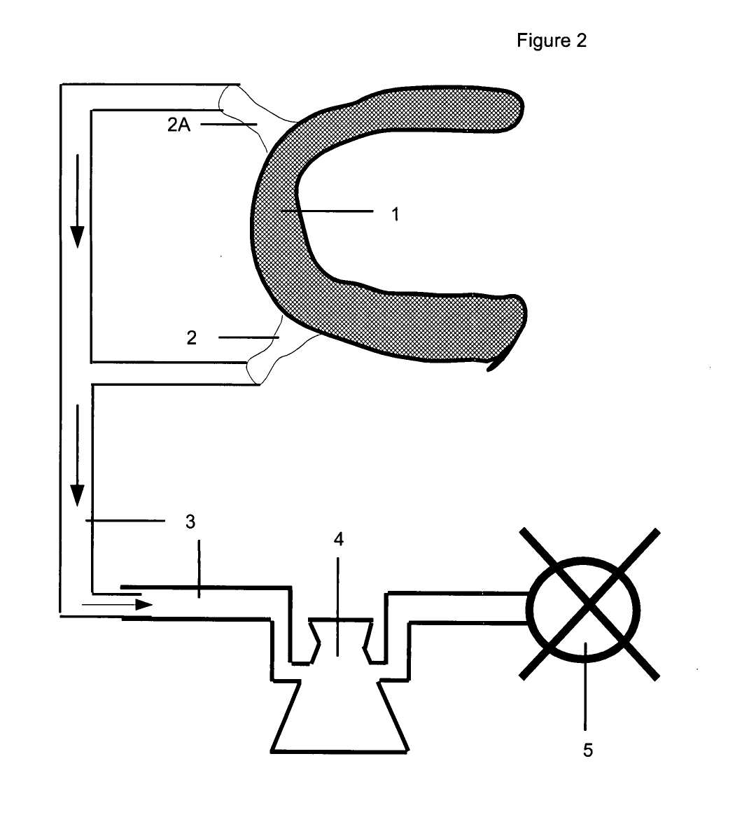

1. A diaper with or without any innovations listed in this application provided with attachment means for one or more vacuum suction lines permitting the attachment of vacuum conduits leading via an optional storage reservoir to a self-contained or central institutional fixed vacuum source for the purpose of inducing air movement to ventilate the diaper and to assist in evacuating contents

2. (canceled)

3. (canceled)

4. The provision of an in-line or parallel storage container for the purpose of and shaped to permit the accumulation of evacuated fluids or wastes with minimal impedance to the transfer of vacuum from the vacuum source to the diaper, said storage container modularly attached to or integrated either into the external surface of the diaper or at any point outside of the absorptive matrix of the diaper of claim 1

5. The integration of the diaper and vacuum circuit (including power source and vacuum generator) of claim 1 into a unitary package, if so desired

6. (canceled)

7. The provision of means to detect and monitor humidity level within the diaper assembly of claim 1

8. The provision of means to integrate charcoal, other odor absorbent means, antibiotics or buffers such as sodium bicarbonate either into the absorbent matrix, or into the structure of the diaper assembly of claim 1

9. The term “DRYPER” to refer to the technology of claim 1 and to be excluded from application to embodiments not consistent with this technology

10. A modular embodiment of the various units of the assembly of claim 1 permitting rapid detachment of storage reservoir, power source and vacuum generator from the diaper of claim 1, if so desired

11. The integration of the self-contained vacuum circuit assembly of claim 1, consisting of a power source and vacuum generator, into a modular unit that can be completely detached and transferred to a new diaper, if so desired.
